SEQUENCE LISTING (12 sequences)

SEQ ID NO: 1 - protein, 165 aa, Homo sapiens
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 2 - protein, 165 aa, Homo sapiens [sequence omitted]

SEQ ID NO: 3 - protein, 20 aa, Homo sapiens
CKKSIENAKQDLLQLKNVIS

SEQ ID NO: 4 - protein, 920 aa, Homo sapiens
MTGYTMLRNGGAGNGGQTCMLRWSNRIRLTWLSFTLFVILVFFPLIAHYYLTTLDEADEAGKRIFGPRVGNELCEVKHVLDLCRIRESVSEELLQLEAKRQELNSEIAKLNLKIEACKKSIENAKQDLLQLKNVISQTEHSYKELMAQNQPKLSLPIRLLPEKDDAGLPPPKATRGCRLHNCFDYSRCPLTSGFPVYVYDSDQFVFGSYLDPLVKQAFQATARANVYVTENADIACLYVILVGEMQEPVVLRPAELEKQLYSLPHWRTDGHNHVIINLSRKSDTQNLLYNVSTGRAMVAQSTFYTVQYRPGFDLVVSPLVHAMSEPNFMEIPPQVPVKRKYLFTFQGEKIESLRSSLQEARSFEEEMEGDPPADYDDRIIATLKAVQDSKLDQVLVEFTCKNQPKPSLPTEWALCGEREDRLELLKLSTFALIITPGDPRLVISSGCATRLFEALEVGAVPVVLGEQVQLPYQDMLQWNEAALVVPKPRVTEVHFLLRSLSDSDLLAMIRRQGRFLWETYFSTADSIFNTVLAMIRTRIQIPAAPIREEAAAEIPHRSGKAAGTDPNMADNGDLDLGPVETEPPYASPRYLRNFTLTVTDFYRSWNCAPGPFHLFPHTPFDPVLPSEAKFLGSGTGFRPIGGGAGGSGKEFQAALGGNVPREQFTVVMLTYEREEVLMNSLERLNGLPYLNKVVVVWNSPKLPSEDLLWPDIGVPIMVVRTEKNSLNNRFLPWNEIETEAILSIDDDAHLRHDEIMFGFRVWREARDRIVGFPGRYHAWDIPHQSWLYNSNYSCELSMVLTGAAFFHKYYAYLYSYVMPQAIRDMVDEYINCEDIAMNFLVSHITRKPPIKVTSRWTFRCPGCPQALSHDDSHFHERHKCINFFVKVYGYMPLLYTQFRVDSVLEKTRLPHDKTKCFKFI

SEQ ID NO: 5 - protein, 165 aa, Homo sapiens; identical to SEQ ID NO: 1

SEQ ID NO: 6 - protein, 165 aa, synthetic construct [sequence omitted]

SEQ ID NO: 7 - protein, 165 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMNEDSILAVRKYFQRITLYLKEKKYSPCAWEVVRAEIMRSFSLSTNLQESLRSKE

SEQ ID NO: 8 - protein, 165 aa, synthetic construct [sequence omitted]

SEQ ID NO: 9 - protein, 166 aa, Homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 10 - protein, 165 aa, Homo sapiens
SYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 11 - protein, 144 aa, Homo sapiens [sequence omitted]

SEQ ID NO: 12 - protein, 172 aa, synthetic construct
  REGION 1..172: note = recombinant protein
CYLSRKLMLDARENLKLLDRMNRLSPHSCLQDRKDFGLPQEMVEGDQLQKDQAFPVLYEMLQQSFNLFYTEHSSAAWDTTLLEQLCTGLQQQLDHLDTCRGQVMGEEDSELGNMDPIVTVKKYFQGIYDYLQEKGYSDCAWEIVRVEMMRALTVSTTLQKRLTKMGGDLNSP

(19) United States
(12) Patent Application Publication
(10) Pub. No.: US 2025/0099544 A1
(43) Pub. Date: Mar. 27, 2025

(54) Treatment of Type 1 Diabetes and Other Conditions Using the Gut Microbiome

(71) Applicant: Claresa Levetan, Bryn Mawr, PA (US)

(72) Inventor: Claresa Levetan, Bryn Mawr, PA (US)

(21) Appl. No.: 18/791,091

(22) Filed: Jul. 31, 2024

Related U.S. Application Data
(63) Continuation-in-part of application No. 16/291,207, filed on Mar. 4, 2019.
Application No. 16/291,207 is a continuation-in-part of application No. 15/815,318, filed on Nov. 16, 2017, now abandoned.
(60) Provisional application No. 63/530,476, filed on Aug. 3, 2023.
(60) Provisional application No. 62/793,989, filed on Jan. 18, 2019.

(51) Int. Cl. A61K 38/21; A61K 9/00

(52) U.S. Cl. CPC A61K 38/212; A61K 9/0053; A61K 38/215

(57) ABSTRACT

This invention provides novel compositions and methods of oral therapy for the inhibition of Th17 and its downstream cytokines utilizing Type I interferons and their analogs among patients with disease states characterized by aberrant gut microbiomes. This invention utilizes Th17 levels in the plasma and cerebrospinal fluid along with clinical symptoms as tools for monitoring treatment dosage and treatment duration of Type I interferons and their analogs to inhibit Th17 and its destructive cascade of attack to cells, tissues and organs resulting in improved outcomes and symptomatology, as well as the prevention of disease expression in those at risk for many disease states, some of which have limited therapeutic options.

### CROSS-REFERENCE TO RELATED APPLICATIONS

The present application claims the benefit of priority to U.S. Provisional Application Ser. No. 63/530,476 filed Aug. 3, 2023; and is a Continuation-in-Part (CIP) of U.S. Non-Provisional application Ser. No. 16/291,207 filed Mar. 4, 2019, which claimed priority to U.S. Provisional Application Ser. No. 62/793,989 filed Jan. 18, 2019 and is a Continuation-in-Part (CIP) of U.S. Non-Provisional application Ser. No. 15/815,318 filed Nov. 16, 2017 the disclosures of which are all incorporated herein by this reference.

### REFERENCE TO SEQUENCE LISTING SUBMITTED IN COMPUTER READABLE FORM

The present application contains a Sequence Listing which has been submitted in ASCII format by way of EFS-Web and is hereby incorporated by reference herein in its entirety. The ASCII file was created on Jul. 28, 2024, and is named C.LEV-023_ST25, which is 22.7 kb in size, and which is identical to the paper copy filed with this application.

The present application additionally contains a Sequence Listing which has been submitted in XML format electronically by way of Patent Center and is hereby incorporated by reference in its entirety. The XML file was created on Nov. 20, 2024, and is named clev-023_XML, which is 15,165 bytes in size.

### FIELD OF THE INVENTION

Embodiments of the present invention provide novel compounds and methods for treating and preventing conditions associated with dysbiotic gut microbiomes and include oral therapies, compositions and methods that inhibit the specific and targeted attack by T helper 17 lymphocytes (Th17) to the cells, tissues, and organs. This invention provides compositions and methods for treatment, stabilization of disease states, and reversal of symptomatology, and prevention of expression of diseases that have a common aberrant gut microbiome and include diseases such as Parkinson's Disease, Amyotrophic Lateral Sclerosis, Alzheimer's Disease, Type 1 Diabetes, Macular Degeneration and other rare and common diseases and neoplasms with each disease state characterized by a common dysbiotic gut microbiome.

New to the art, this inventor describes how and why conditions with a common aberrant gut microbiome are also conditions in which T helper lymphocyte 17 (Th17) and its downstream cytokine IL-17 (IL-17) initiate, trigger and/or exacerbate the expression of unique disease states defined by the individual's gut microbiome and that Th17 and IL-17 specifically target and attack cells, tissues and organs of unique disease states defined by the individual's gut microbiome with expression of Th17 dependent on the individual's genome and environmental triggers.

This inventor finds that an individual's gut microbiome imprints on Th17 cells, which are constitutively found in the mucosal surface of the gastrointestinal (GI) tract. This inventor teaches that the impact of the individual's library of gut microorganisms interacts directly with Th17 found in the lamina propria of the gastrointestinal mucosa and forms a Gastrointestinal Immune Memory (GIM) within Th17.

This invention describes the relationship between 1) a normal gut microbiome and a dysbiotic gut microbiomes, which are uniquely characteristic of specific disease states 2) the imprinting of the gut microbiome onto Th17, which is constitutively found in the mucosal surface of the gastrointestinal tract and forms the individual's Gastrointestinal Immune Memory (GIM) within the Th17 cells 3) how dysbiotic gut microbiomes defining common and rare disease states ranging from type 1 diabetes, Parkinson's Disease, Macular Degeneration, Amyotrophic Lateral Sclerosis to many forms of cancer, all of which have unique gut microbiomes defined by a lack of or diminished concentrations of unique microorganisms that is specific to one or more diseases that the individual is at risk for. The interaction between the individual's gut microbiome, which may contain aberrant numbers and concentrations of microorganisms characteristic of one or more diseases, results in the individual's GIM lacking recognition by the individual's immune system that one or more conditions are “self,” with the potential for conditions to manifest based upon the GIM, environmental triggers and the individual's genome. For example, age-related macular degeneration has a unique microbiome characterized by significantly lower levels of Firmicutes and higher levels of Bacteroidota compared to healthy controls (Zheng, Sec. Systems Microbiology, (14) 2023, Zisimopoulos, Ophthalmologica 2021; 244:173-178). Age related macular degeneration rarely occurs before the age of 55 and early macular degeneration is more frequent in men that women. Th17 cells are found to be significantly increased in age-related macular degeneration by its impact on stimulating IL-17 and interferon Gamma. This inventor hypothesizes that an aberrant microbiome that leads to the GIM not recognizing the macula as “self” leads to degeneration of the macula that is Th17-led when an environmental pathogen such as a virus may trigger Th17 and based on the individua's genome may predict age of onset and sex predilection for the immune attack on the macula. The immune attack is therefore misdirected to healthy cells because the GIM does not recognize the macula as “self” because of the aberrant gut microbiome imprinted on Th17.

This inventor finds that a specific and targeted attack to the brain, heart, eyes, pancreas and elsewhere are defined by the individuals aberrant gut microbiome leading to lack of recognition of self of many common and rare conditions, and the clinical expression may be triggered by the environment and impacted by the patient's genome.

This inventor finds the triggers to disease expression to include external pathogens, the individual's genotype, the gastrointestinal immune memory (GIM), and other outside triggers, which include chronic stress that can activate the hypothalamic-pituitary-adrenocortical axis to promote the release of glucocorticoids and ultimately result in the individual being at higher risk of cell and organ damage due to an attenuated response to tissue glucocorticoids from chronically elevated glucocorticoids.

The Gastrointestinal Immune Memory of the conditions associated with aberrant gut microbiomes result from an immune attack to cells, tissue and organs of conditions defined by an aberrant gut microbiome because the full library of normal microbiota is not present in the microbiome and those conditions with atypical microbiomes are not recognized as “self” when Th17 is expressed to protect from another attack such as cold, flu, COVID-19 etc. The Gastrointestinal Immune Memory does not form a memory of such conditions being “self” because of decreased and/or altered numbers, concentrations and ratios of specific microorganisms associated with each disease state having a common aberrant microbiome.

For example, following COVID-19, many other conditions have been shown to be expressed for the first time ranging from type 1 diabetes, Parkinson's disease, recurrent Multiple Sclerosis and other conditions with characteristic gut microbiomes, in which Th17 is etiologic to the attack to the beta cells of the pancreas in type 1 diabetes or targeted to parts of the brain in Parkinson's Disease (substantia nigra) and the white matter in Multiple Sclerosis.

This inventor also teaches that a specific and targeted Th17-led attack is initiated from the gut mucosa into the interstitium into the bloodstream to different organs and through a variety of neural pathways to the brain.

In humans, the gut microbiome is established within the first few years of life, by which time, the immune system has developed an immune barrier that is based upon the gut microbiome presented to Th17. The normal immune barrier occurs when the normal gut microbiome trains Th17 to recognize the normal gut microbiome as “self.” When the gut microbiome does not have the complete amounts, ratios and concentrations of microorganisms found in healthy gut microbiomes, Th17 does not have all necessary microorganisms to properly imprint upon the immune system and the Gastrointestinal Immune Memory does not see disease states characterized by aberrant gut microbiomes as “self.”

Many disease states, even rare conditions and those with few treatment options such as Amyotrophic Lateral Sclerosis, Parkinson's Disease, type 1 diabetes, and other conditions, have a common aberrant microbiome that are characteristic for the disease state. One individual may be at risk for a number of conditions based upon their gut microbiome.

This inventor finds that a healthy gut microbiome specifically teaches Th17 to recognize the vast ecosystem of microorganisms in the normal gut microbiome as “self” and creates a Th17 immune memory does not to attack the body's own cells, tissues, and organ systems when Th17 is triggered to begin an immune attack against a foreign antigen, such as with an infection.

For many decades the term autoimmune disease had a much narrower definition, as a condition in which self-destructive immune cells attack one's own healthy cells. In this invention, autoimmune diseases include diseases that have yet to be characterized as autoimmune but include diseases in which the etiology of the cell destruction is due to a Th17-led attack based upon the individual possessing a common aberrant gut microbiome common to that disease state.

New to the art, this invention finds that each disease state or condition with an aberrant gut microbiome results from a Th17-led attack and may include downstream cytokines IL-17, IL-21, IL-22, IL-23, IL-6 and TNF-alpha. The Th17 attack is specific and targeted to the cell, tissue or organ characterized by the aberrant gut microbiome. For example, even in conditions like Schizophrenia, Th17 and its downstream cytokines have been shown to be etiologic (Debnath, Schizophrenia Bull. 2014 November; 40 (6): 1412-1421) with Th17 traveling from the mucosal lining of the GI tract into the interstitial fluid and into the bloodstream where it triggers interstitial neurons and travels to and disrupts the blood-brain barrier, infiltrates the central nervous system, and, along with other cytokines leading to neuroinflammation. Schizophrenia has been shown to have a characteristic gut microbiome deficient in microorganisms that are not seen deficient in those without Schizophrenia (Severance, Curr Top Behav Neurosci. 2020; 44 (67-84).

Individuals may be at risk for one or more disease states during their lifetime and possess multiple aberrancies in their gut microbiome. This invention finds that external triggers, such as an infection may lead not only to attack by Th17 to the organs of the infections, but also lead to disease because the GIM is not reading that disease state as self.

New to the art, this invention demonstrates that each condition with a characteristic gut microbiome is a condition in which Th17 leads a specific and targeted attack and Th17 is etiologic in the expression of the disease state. This inventor teaches that when the gut microbiome imprints on Th17 in the mucosal surface of the gastrointestinal tract, there is the formation of a Gastrointestinal Immune Memory. When there are aberrant gut microbiomes that are associated with a specific disease, and the disease state has a characteristic loss of microorganisms or altered concentrations of microorganisms as compared to the normal healthy gut microbiome, and the absence or/or altered concentrations of microorganisms imprinting on Th17 forms the Gastrointestinal Immune Memory, which does not recognize the disease state associated with an aberrant gut microbiome as “self.”

This inventor finds the triggers to disease expression to include external pathogens, the individual's genotype, the gastrointestinal immune memory (GIM), and other outside triggers, which include chronic stress that can activate the hypothalamic-pituitary-adrenocortical axis to promote the release of glucocorticoids and ultimate result in the individual being at higher risk of cell, and organ damage due to an attenuated response to tissue glucocorticoids from chronically elevated glucocorticoids.

More recently, Th17 has been found to cause chronic inflammation in age-related macular degeneration. Th17 cells (Chen, Cell Physiol Biochem 2017; 44:357-367), and age-related macular degeneration is associated with a common gut microbiome (Zisimopoulos, Ophthalmologica 2021; 244:173-178).

This invention provides new compositions, methods, and formulations of oral Type I interferons and their analogs to specifically inhibit Th17 and IL-17 receptors in the mucosal surfaces of the gastrointestinal tract where Th17 and IL-17 are constitutively found. Th17, while in the lamina propria of GI tract, expands in number, triggers IL-17 and both travel from the mucosal surface through the interstitial surface into the blood stream and to the specifically targeted cells characterized by a unique and aberrant gut microbiome associated with specific disease(s).

By inhibiting Th17 at its source, the inhibition of IL-17 and other downstream cytokines occur including but not limited to IL-21, IL-22, IL-23, IL-6 and TNF-alpha, which migrate from the mucosa of the GI tract into the interstitial fluid where Th17 and IL-17 stimulate other cytokines to attack organs and cells, including the central nervous system (FIG. 1).

This inventor has uniquely found that oral therapies that specifically inhibit Th17 lead to better outcomes with slower progression of disease among conditions defined by aberrant gut microbiomes ranging from type 1 diabetes to idiopathic pulmonary fibrosis and graft vs. host disease.

The gut microbiome forms and is modified during the first decade of life but remains malleable throughout life and can change due to several factors including antibiotic therapy. During these first years of life, the gut microbiome-dependent imprints onto Th17 memory cells in the mucosa of the GI tract (Kumar, Immunity. 2016 Mar. 15; 44 (3): 659-671, Pandiyan, Front. Immunol., 8 Mar. 2019).

This invention finds that individuals lacking a complete “normal” gut microbiome and therefore having an aberrant gut microbiome that is characteristic for one or more disease states, have the potential to develop these diseases during their lifetime. This inventor finds that each condition with a characteristic dysbiotic gut microbiome can manifest and express the disease when Th17 is triggered to attack by an external factor, such as an infection in combination with other factors because the GIM does not see those diseases with aberrant gut microbiomes as “self”. Lack of presentation of healthy concentrations, numbers and ratios of microorganisms leads to lack of Th17 recognizing conditions associated with dysbiotic microbiomes as “self” and poses increased risk for a Th17-initiated attack to cells, tissues and organs defined by the aberrant gut microbiome.

Other contributors to Th17 attacking cells associated with conditions of aberrant gut microbiomes include the age, sex, and genotype of the individual. For example, Parkinson's affects twice as many men than women and occurs predominately among people aged 60 years or greater. All these factors contribute to whether a Th17 attack to cells will occur.

One of the biggest triggers of Th17 is external infections in which Th17 and its cytokine cascade are upregulated. For example, in COVID-19, in which Th17 is triggered from the gut and lead to systemic attack, many other conditions and diseases with characteristic gut microbiomes have been found to occur following COVID-19, including type 1 diabetes, Graves' Disease, Multiple Sclerosis relapses and many other conditions with aberrant gut microbiomes.

This inventor finds that when Th17 is upregulated by COVID-19 and the GIM does not recognize other disease states(s) as “self” due to the individual's aberrant gut microbiome, Th17 may also attack other cells associated with such disease states. For example, Parkinson's Disease has been well described to occur following COVID-19. Parkinson's is a condition with a unique gut microbiome and is a condition in which Th17 is etiologic in cell destruction in the substantia nigra cells of the brain and has been shown to occur following COVID-19 infections (Li, Journal of Translational Medicine 18, 4932, 2020).

### BACKGROUND OF THE INVENTION

This invention relates to the new oral compositions, formulations, usage, dosage and treatment using Type I interferons and their analogs for oral inhibition of Th17, where Th17 is constitutively found and expressed and is a trigger for conditions and diseases, including autoimmune diseases, many types of infections and conditions that have characteristic gut microbiomes, including neoplasms characterized by having a T helper lymphocyte-17 (Th17)-led attack to cells, tissues and organs.

This invention specifically finds that inhibition of Th17 prevents IL-17 expression in the gastrointestinal tract before they trigger other cytokines that emerge to target organs including the brain.

This invention presents studies conducted using oral therapies for a variety of conditions prior to discovery of Th17 and IL-17 and prior to the understanding that Th17 and IL-17 are constitutively found in the mucosa of the gastrointestinal tract.

Specifically, this invention finds that Type I interferons and their analogs are effective because Type 1 Interferons and their analogs are stable down to pH of 2.0, thus are not broken down or degraded in the stomach (Utsumi. Biochim Biophys Acta. 998 (2): 167-172 (1989).

Oral Type I interferons and their analogs are not absorbed systemically when delivered orally and are not associated with systemic immune suppression, infection, or neoplasms as is the case with Type I interferons when delivered parentally in dosages 10,000-100,000-fold higher than the dosages presented in this invention when delivered orally. This invention has also found that in human trials, prior to understanding the mechanism of action of Type I interferons delivered orally and how they could delay and prevent disease, that dosages above 10,000 IU were not beneficial and did not demonstrate benefit, which this invention has found due to conformational changes at the Th17 receptor when higher dosages have been used and low dosages of oral Type I interferons and their analogs reduce Th17 and IL-17 levels in the plasma and/or cerebrospinal fluid or at the site of attack.

This invention provides novel compositions, usage, methods, dosages for oral delivery of Type I interferons to treat, limit and inhibit disease activity and prevent condition(s) and disease(s) with a common and characteristic gut microbiome for specific diseases which possess common deficiencies and aberrations in the numbers and types of gut microorganisms as compared to that of those with healthy gut microbiomes.

Type I interferons have a unique property of withstanding a pH of 2.0 allowing it to pass through the GI tract and not degraded by stomach acid (Utsumi. Biochim Biophys Acta. 998 (2): 167-172 (1989). Additionally, dosages of 108 IU of oral interferon beta were given to human subjects and failed to demonstrate measurable plasma levels (Witt, Journal of Interferon Research 12:411-413 (1992).

This inventor describes oral treatment with Type I interferons and their analogs to inhibit Th17 egress from the GI mucosa to limit disease expression and symptomatology for those who possess one or more aberrant gut microbiomes that are characteristic and specifically seen with conditions ranging from neurological conditions including, but not limited to Amyotrophic Lateral Sclerosis, Parkinson's Disease, Multiple Sclerosis, Acute Schizophrenia, Autism Spectrum Disorder, Alzheimer's disease, Macular Degeneration, cancers, including cholangiocarcinoma, breast and colon cancer which are now found to be conditions which there is a Th17-led attack to the specific condition.

Given the significant risk for systemic and untargeted immunosuppression, including risk for viruses that among many conditions, a fatal disease known as progressive multifocal leukoencephalopathy (PML), which is uniquely seen among patients on immunosuppressants, which occurs in 4000 Americans annually, the ability to have a method and monitoring disease and adapt dosage based on Th17 levels may reduce risk of many other serious side effects associated with systemic immunosuppressants.

Type I interferons have been shown to inhibit Th17 (Guo, J Clin Invest 2008; 118 (5): 1680-1690), including alpha interferons (Moschen, Immunology 2008, (213): 9-10 (779-787), beta interferons (Ramgolam J Immunol. 2009, 15; 183 (8): 5418-27, gamma interferons (Yeh, J Neuroimmunol 2014; 267 (0): 20-27 and tau interferons (Ren, Mediators of Inflammation 2016 (2839232) with beta interferons demonstrating the best inhibition of Th17.

This invention provides for oral Type I interferon and analog therapy as a means of inhibiting Th17 when it is expressed for the purposes of autoimmune attack by inhibiting Th17 with treatment at its source using oral Type I interferons and their analogs because Th17 control occurs in the small intestine therapy (Esplugues. Nature. 475 (7357): 514-8 (2011).

Previously, it has been demonstrated that intestinal Th17 cells correlates with disease activity in multiple sclerosis (Cosorich, Science Advances 12 (2017): 3; 7, e1700492). Movement of Th17 into the interstitial tissue to other organs has been shown by Krebs. Immunity. 45 (1078-1092) (2016) and when Th17 is triggered, it leaves the intestinal wall into the interstitial fluid and enters the blood stream and/or communicates with interstitial neurons and targets a specific organ including the brain.

This inventor demonstrates the link between conditions at risk for a specific and targeted Th17 attack as a result of the GIM not recognizing a disease state as “self” resulting in an attack to an organ, tissue or cells, which Th17 sees as foreign and leading to specific and targeted cellular tissue or organ attack when there is an environmental trigger such as an infection, combined with genotype leading to a Th17 attack and Th17 also attacks an organ, tissue or cells of a distinct condition based upon its lack of recognition as “self” a disease state defined by its characteristic dysbiotic gut biome.

In addition, this inventor posits that not only is there a relationship between an environmental trigger, the GIM and the Th17 attack to an organ based on the but microbiome, but also the genotype plays a role in Th17 disease expression. For example, Multiple Sclerosis is typically seen in women with an onset of age between 20-40 years, while Parkinson's is seen in men 1.5 times higher than in women and most often occurs after the age of 50, while both Schizophrenia and type 1 diabetes, which have unique gut microbiomes and Th17 has been found etiologic, occurs most often in younger populations.

Many conditions have found to be expressed in those who have had COVID-19, which are conditions that have a common gut microbiome that is deficient in the same microorganisms and are conditions in which Th17 has recently been considered etiologic including, but not limited to: Type 1 Diabetes, Parkinson's Disease, Alzheimer's Disease, Rheumatoid Arthritis, Systemic Lupus Erythematosus, Autoimmune Hemolytic Anemia, Autoimmune Thrombocytopenia, Guillain-Barré syndrome, Multiple Sclerosis and others.

This inventor finds:

Th17 has roles including having potent proinflammatory properties and plays a major role in many acute and chronic inflammatory and chronic disease states, including cancers and graft vs. host vs. disease.

Although Th17 was initially thought to be produced in peripheral lymphoid organs or generated in thymus (Langrish, 2005; Harrington et al., 2005; Park et al., 2005), the laminal propria within the mucosal surface of the gastrointestinal and pulmonary lining is a key region that Th17 as a proinflammatory is constitutively found and modulated (Mucida, J Clin. Immunol 2009 Allergy May; 123 (5): 997-1003).

Th17 cells secrete other signature cytokines, including IL-17, whose receptors are also found in the lamina propria layer of the mucosa of the GI tract and IL-17 is induced by Th17 in the lamina propria (Esplugues, Nature. 2011, 475 (7357): 514-8

This inventor has found that the prior human studies with oral interferons occurred prior to both the discovery of Th17 and the association between T17 as etiologic in the destruction in many disease states and prior to the association of Th17 diseases with characteristic gut microbiomes.

This inventor finds Type I oral interferon alpha and beta and their analogs are excellent inhibitors of Th17 with beta interferon being the best inhibitor of Th17 and may provide direct inhibition of Th17 rather than a systemic approach to treatment.

This inventor finds that because it was not previously understood that Th17 is constitutively found in the GI tract and that oral interferon withstand a pH of 2.0, and the previous data demonstrating that no positive effects were seen when oral Type I interferons were given in dosages above 10,000 IU daily and usage of oral Type I interferons and their analogs have not been considered for usage in diseases with altered gut microbiomes that are Th17 associated disease states.

This s inventor finds that delivery of Type I interferons directly to Th17 receptors in dosages higher than 10,000 IU results in an allosteric conformational change to the Th17 receptor that impacts the binding of oral interferon to the orthosteric Th17 binding sites. For example, in new onset type 1 diabetes, there was a significant preservation of insulin-producing beta cells at one year among children and adults who received oral interferon in a dosage of 5000 IU daily of oral compared to a placebo. In those randomized to 5000 IU daily, at one year, the treatment group receiving 5000 IU oral interferon daily the placebo group lost 29% of their endogenous insulin secretion compared to a 54% loss of endogenous insulin secretion at one year in the placebo arm, while those on higher dosages had no significant differences insulin secretion than placebo. Adverse events occurred at similar rates in all treatment groups (Rother Diabetes Care. 2009; 32 (7): 1250-5). At the time of this study, neither the association with Th17 as etiologic in the attack on insulin-producing beta cells had been made nor was the gut microbiome association with Type 1 Diabetes known.

As with the genome, there are multitudes of microorganisms forming the GIM and each person has a unique GIM based on the numbers, missing or diminished types and combinations of microorganisms in their gut microbiome. The GIM has a faltered memory for conditions with missing and diminished microorganisms and does not recognize those medical conditions associated with specific aberrant gut microbiomes as “self.”

For example, type 1 diabetes is often clinically expressed after a viral illness. Type 1 diabetes is a disease characterized by a gut microbiome with reduced numbers of Lactobacillus, Bifidobacterium, Akkermansia, Roseburia faecis, Faecalibacterium prausnitzii and Bacteroides (Zheng Diabetes Metab Res Rev. 2018; 34 (7): e3043). Th17 has now been found to be etiologic in the attack on beta cells within the pancreas resulting in the onset of type 1 diabetes with elevated levels of Th17 found in the bloodstream and pancreas of patients with new onset type 1 diabetes, as well as in the GI mucosa.

Another example in Parkinson's Disease, Prevotellaceae is found to be reduced by 77.6% as compared with controls (Scheperjans et al, Mov Disord. 2015; 30 (3): 350-8) and Th17 leads the neuronal attack resulting in the lack of dopamine production in the basal ganglia and substantia nigra of the brain (Shukla et al, Mov Disord. 2018; 33 (10): 1560).

Based on the findings that Th17 and IL-17 are constitutively found in the mucosal layer of the gastrointestinal tract, this inventor finds that oral usage of oral Type I interferons and their analogs that have previously been used systemically to inhibit the Th17 and IL-17 and thus inhibiting the downstream cytokines that they trigger including IL-21, IL-22, IL-23, IL-6 and TNF-alpha, may be used orally in different dosages when targeting the Th17 receptors directly with an oral Type I interferon or an oral interferon analog.

Previously, inhibitors of Th17 and immune agents have been given systemically targeting Th17 and IL-17, after they have migrated from the gut to the organ of attack (brain in the case of Parkinson's Disease or pancreas in the case of type 1 diabetes).

FIG. 1 demonstrates a novel approach using oral treatment of diseases with dysbiotic gut microbiomes. Immunotherapies inhibiting Th17 and its downstream cytokines are dosed systemically to inhibit Th17 after it has already begun the attack on an organ, tissue, or cells. This invention inhibits Th17 directly within the gastrointestinal system where Th17 is formed, found, and migrates to the organs of attack utilizing oral Type I interferons and their analogs.

Until now, there are no specific and targeted compositions for the treatment and prevention of diseases with aberrant gut microbiomes, nor are compositions for oral treatments that directly inhibit Th17 in the GI tract resulting in an inhibition of the downstream cytokines stimulated by Th17. There have only been systemic therapies aimed at treating Th17, which reflects why much higher systemic dosages of Type I interferons have been required to treat Th17 in contrast to treatment directly to the Th17 in the GI tract.

Because there is a circadian rhythm in the expression of Th17, with production of Th17 highest at midnight and lowest at noon, therefore this inventor recommends usage interferons at bedtime rather than in the morning. New to the art of clinical practice of prescribing interferon therapy is directing patients to use interferon therapy preferentially at bedtime given the peak levels of Th17 are at midnight. This therapy provides a different approach, which may obviate or be used in conjunction with the usage of systemic monoclonal antibodies to treat some of the most vexing disease states that there are currently limited treatment options.

### DESCRIPTION OF THE RELATED ART

Weaver (U.S. Pat. No. 9,017,685) generally describes inhibiting the generation of Th17 by exposing a Th17 effector cell precursor to an antagonist of interleukin-23 (IL-23) function thereby inhibiting the generation of the Th17 effector cell.

This inventor describes specific methodology of identifying a disease state by symptomatology, gut microbiome, plasma, and serum markers and by elevated measurements of Th17 in the plasma and/or serum with specific selection of an interferon for treatment with dosage, mode of delivery and monitoring of disease and changes in treatment dosage based upon Th17 levels, symptomatology, and course of disease.

For example, when a patient with new onset type 1 diabetes is found to have a gut microbiome consistent with patients with type 1 diabetes and elevated Th17 levels in the plasma and/or serum, interferon alpha 2a or interferon Beta 1a or analogs of these interferons may specifically be selected for daily usage in the patient and given at a maximum of 5000 IU orally daily along with other medications including insulin. This inventor has found that in man, the association of oral interferon dosages above 5000 IU result in a conformational change to the Th17 receptor rendering inactivity in the effect of inhibition of Th17. After 4 weeks of oral interferon therapy, repeat levels of Th17 levels in the plasma or serum will be evaluated. Dosage of insulin and other diabetes medications may begin to be reduced based upon levels of Th17.

Whereas Deykin (U.S. Pat. No. 10,500,254) describes a methodology of more rapidly providing a dose escalation of interferon alpha-1b intramuscularly to patients with Multiple Sclerosis to reduce flu-like symptoms, Deykin does not provide for dose determination and escalation based upon baseline and follow-up Th17 levels. Given the side-effect profile of interferon alpha-1b and the systemic immunosuppression potential, this inventor provides a new methodology of utilizing the lowest dosages of interferon alpha-1b for a condition such as Multiple Sclerosis (MS) using the patient's Th17 levels for monitoring and determining if a reduction in dosage may be appropriate, which Deykin does not describe.

Typically, interferon beta is used in the treatment of multiple sclerosis and the dosage is given parentally with standard titration each week with the dosage increased weekly for 4 weeks without examination or titration based upon Th17 levels or symptomatology of disease. New to the art, in this invention, is the specific monitoring of Th17 levels on a regular basis for the initiation of oral interferon to determine efficacy along with symptomatic improvement in the disease state treated with a maintenance found at the dosage in which the patient has diminished levels of Th17 with improved symptomatology.

Many studies have shown that even at dosages of oral interferon of 10⁵ IU orally interferon was not found in plasma, no absorption is found in the plasma or shown to affect white blood cells (Eid, Journal of Interferon and Cytokine Research 19: 157+169 (1999), Wills J. Interferon Res. 1984, 4, 399-409, Gibson. J. Interferon Res. 1985, 5, 403-408).

Whereas Stimson (U.S. Pat. No. 10,487,127) specifically suppresses Th2/Th17-mediated immune response for a said disease like cancer or allergy using a hybrid of IFN-α10 and IFN-α14 to bind to the interferon receptors, Stimson does not evaluate and treat based upon plasma or serum or plasma levels of Th17. In U.S. Pat. No. 9,522,173, Stimson provides enhancement of a Th1-mediated immune response and suppression of a Th2/Th17-mediated immune response selected from IFN-α10, IFN-α14, and a hybrid thereof for treating of an autoimmune disease, an inflammatory disease (e.g., inflammatory bowel disease) and allergy or an associated allergic condition. Stimson neither utilizes measurement of Th17 in the plasma or serum, nor does Stimson measure baseline Th17 levels in for the selection of the interferon, dosage of the interferon, or make dosage changes and modification based upon Th17 levels.

Levetan (United States Patent Application 20190328839) has described evaluating the patient's gut microbiome for usage of Th17 inhibitors but does not utilize measurement of Th17 levels for selection of the interferon, dosage, method of delivery and dose modification, as described herein.

### BRIEF SUMMARY OF THE INVENTION

This invention relates to compositions and methods for usages, dosing and delivery of Type I oral interferons and their analogs to children, adults and other mammals for the treatment of diseases associated with elevated plasma, serum and/or cerebrospinal levels of T lymphocyte 17 (Th17) or its downstream cytokines such as Interleukin 17 (IL-17), including all diseases characterized by a common gut microbiome for that specific disease and that is a condition in which Th17 leads the autoimmune destruction. This includes diseases that have not been considered autoimmune, but which have aberrant gut microbiomes characteristic of the disease states and range from common diseases such as age-related macular degeneration to more rare diseases such as Amyotrophic Lateral Sclerosis and a variety of cancers. This novel approach may prevent, limit or reverse clinical symptoms of many conditions with limited therapies and may obviate or be used in conjunction with systemic monoclonal antibodies by inhibiting T helper 17 and Interleukin-17 at their source. This inventor uses oral Type I oral Interferons and their analogs as inhibitors of Th17 and IL-17.

This inventor describes methods for using Type I interferons and their analogs orally for conditions, including those with limited treatment options, which have elevated Th17 levels, using interferons for selectively inhibiting Th17 and reducing levels and the Th17-initiated specific and targeted attack to cells, tissues and organ systems based upon an individual's gut microbiome, with compositions, dosages and delivery of Type I interferons and their analogs for treatment with duration of treatment and dosage based upon the plasma, serum and/or cerebrospinal levels of Th17, the disease state, disease severity and symptomatology. This invention also includes compositions methods, dosage regimen and modality of delivery for usage of Type I interferons for prevention of disease states associated with elevated Th17 levels with interferon selection, modality and dosage based upon the individual patient's family history, genotype, increased risk for disease states, levels of Th17, symptomatology and the patient's gut microbiome.

Weaver (U.S. Pat. No. 9,017,685) describes inhibiting the generation of Th17 by exposing a Th17 effector cell precursor to an antagonist of interleukin-23 (IL-23) function thereby inhibiting the generation of the Th17 effector cell.

This inventor describes specific methodology of identifying a disease state by symptomatology, gut microbiome, plasma, and serum markers and by elevated measurements of Th17 in the plasma and/or serum with specific selection of an interferon for treatment with dosage, mode of delivery and monitoring of disease and changes in treatment dosage based upon Th17 levels, symptomatology, and course of disease. For example, when a patient with new onset type 1 diabetes is found to have a gut microbiome consistent with patients with TID and elevated Th17 levels in the plasma and/or serum, interferon alpha 2a or interferon Beta 1a or analogs of these interferons may specifically be selected for daily usage in the patient and given at a maximum of 5000 IU orally daily along with other medications including insulin. After 4 weeks of oral interferon therapy, repeat levels of Th17 levels in the plasma or serum will be evaluated. Dosage of insulin and other diabetes medications may begin to be reduced based upon levels of Th17.

Whereas Deykin (U.S. Pat. No. 10,500,254) describes a methodology of more rapidly providing a dose escalation of interferon alpha-1b intramuscularly to patients with Multiple Sclerosis to reduce flu-like symptoms, Deykin does not provide for dose determination and escalation based upon baseline and follow-up Th17 levels. Given the side-effect profile of interferon alpha-1b and the systemic immunosuppression potential, this inventor provides a new methodology of utilizing the lowest dosages of interferon alpha-1b for a condition such as Multiple Sclerosis (MS) using the patient's Th17 levels for monitoring and determining if a reduction in dosage may be appropriate, which Deykin does not describe.

Typically, interferon beta is used in the treatment of multiple sclerosis and the dosage is given parentally with standard titration each week with the dosage increased weekly for 4 weeks without examination or titration based upon Th17 levels or symptomatology of disease. New to the art, in this invention, is the specific monitoring of Th17 levels on a regular basis for the initiation of oral interferon to determine efficacy along with symptomatic improvement in the disease state treated with a maintenance found at the dosage in which the patient has diminished levels of Th17 with improved symptomatology.

Many studies have shown that even at dosages of oral interferon of 105 IU orally interferon was not found in plasma, no absorption is found in the plasma or shown to affect white blood cells (Eid, Journal of Interferon and Cytokine Research 19: 157+169 (1999), Wills J. Interferon Res. 1984, 4, 399-409, Gibson. J. Interferon Res. 1985, 5, 403-408).

Whereas Stimson (U.S. Pat. No. 10,487,127) specifically suppresses Th2/Th17-mediated immune response for a said disease like cancer or allergy using a hybrid of IFN-α10 and IFN-α14 to bind to the interferon receptors, Stimson does not evaluate, and treat based upon plasma or serum or plasma levels of Th17. In U.S. Pat. No. 9,522,173, Stimson provides enhancement of a Th1-mediated immune response and suppression of a Th2/Th17-mediated immune response selected from IFN-α10, IFN-α14, and a hybrid thereof for treating of an autoimmune disease, an inflammatory disease (e.g., inflammatory bowel disease) and allergy or an associated allergic condition. Stimson neither utilizes measurement of Th17 in the plasma or serum, nor does Stimson measure baseline Th17 levels in for the selection of the interferon, dosage of the interferon, or make dosage changes and modification based upon Th17 levels.

Levetan (United States Patent Application 20190328839) has described evaluating the patient's gut microbiome for usage of Th17 inhibitors but did not utilize measurement of Th17 levels for selection of the interferon, dosage, method of delivery and dose modification, as described herein.

Because, to date, there are no medical indications for oral interferons or their analogs, reference is now made to methods of delivery of oral Type I interferons and their analogs. Embodiments of this invention are specifically for oral delivery of alpha, beta, gamma and tau interferons and their analogs that over the past 40 years have only been used for parental usage with no oral usages for medicinal purposes in man. Embodiments include formulations, which consider taste, smell, color, and texture and include and are not limited solutions, suspensions, syrups, capsules, and disintegrating oral preparations.

### BRIEF DESCRIPTION OF THE DRAWINGS

FIG. 1 is an illustration demonstrating the stimulation of Th17 by the environment and how treatment of with an oral Th17 inhibitor limits Th17 and its downstream cytokines to cells and tissues of the disease state targeted.

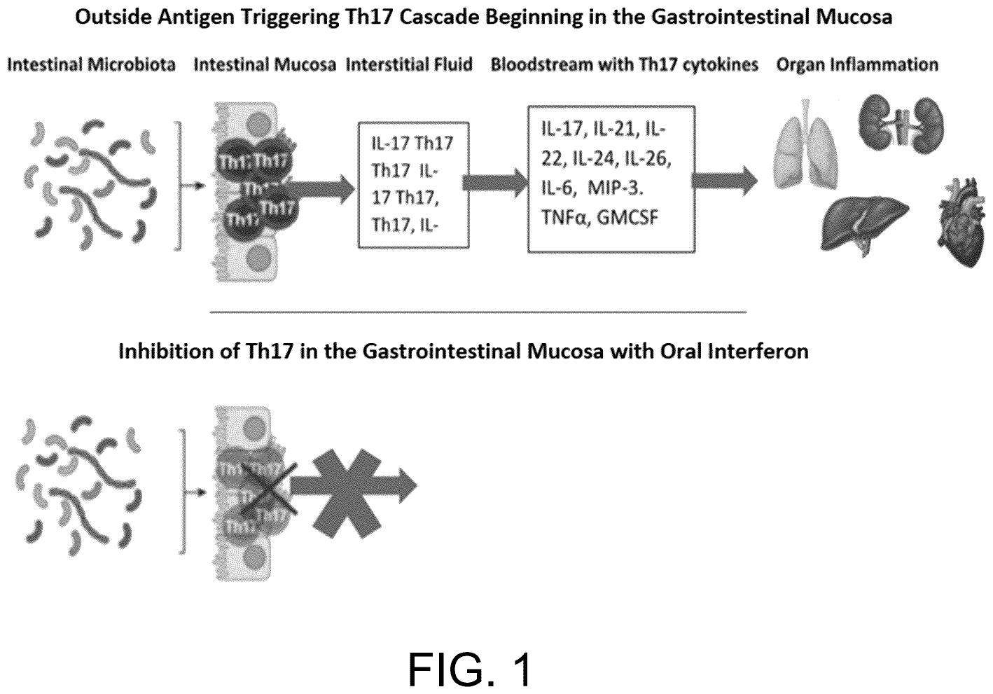

FIG. 2 is a representation of PEGylated Interferon Alfa 2a and Type 1 Interferon Receptor.

FIG. 3 is a representation of the amino acid sequence of Interferon Alpha 2A with lysine residues highlighted.

FIG. 4 is an illustration of two different modifications of lysine side chains.

### DETAILED DESCRIPTION

The present disclosure relates to new compositions, methods, and formulations of oral Type I interferons and their analogs to specifically inhibit Th17 and IL-17 receptors in the mucosal surfaces of the gastrointestinal tract where Th17 and IL-17 are constitutively found. The methods include measuring Th17 in the serum and plasma of a human patient or non-human mammal patient suffering from conditions associated with aberrant gut microbiomes, determining whether Th17 is elevated in the patient, and treating the patient with an oral Th17 inhibitor such as an oral Type I interferon to inhibit a source of Th17 present in the gastrointestinal tract of the patient. The methods further include monitoring the patient for symptoms of the disease as well as the Th17 level in the serum or plasma of the patient, and/or cerebrospinal, tissue or cellular levels of the patient, and adjusting the treatment with the oral Type I interferon based on the monitoring.

The Type I interferons can include alpha, beta, gamma and tau interferons and their analogs. The Type I interferons can be used orally in man and other mammals in dosages of less than 10,000 IU per day as therapy for limiting the symptomatology, morbidity and mortality of the said conditions associated with aberrant gut microbiomes and characterized by diseases in which Th17 is etiologic in cell, tissue, and organ damage.

The Type I interferons including alpha, beta, gamma and tau interferons and their analogs can be specifically used in man and other mammals and used orally in dosages of less than 10,000 IU per day as therapy for preventing the onset of diseases and conditions associated with aberrant gut microbiomes and characterized by diseases in which Th17 is etiologic in cell, tissue, and organ damage.

The analogs to Type I interferons can include novel changes and modifications to the amino acid structure of Type I interferons that maintain its biological effect when delivered orally for the specific usage of inhibiting Th17 in man and other mammals for the purposes of prevention of disease expression, limiting disease progression, disease symptomatology and prevention mortality and morbidity of disease in conditions associated with aberrant gut microbiomes where Th17 is etiologic, and therapy is used for inhibition of Th17. The analogs can include, but are not limited to lipidation of one or more amino acids with introduction of a palmitoyl group, such as lipidation of lysine residues by palmitic acid or aspartic acid or glutamic acid residues by 16-aminopalmitic acid or introduction of carboxyl group or introduction of an amino alkyl group. The analogs can also include pegylated forms of the Type I interferons. The Type I interferons can be modified by other methods of protein modification to improve the biopharmaceutical properties of proteins include site-selective chemical modification such N-terminal acetylation and a C-terminal amidation, addition of an N-terminal cysteine, and cyclization.

Methods of treating or preventing conditions and diseases in man and other mammals having aberrant gut microbiome(s) that are characteristic of one or more specific disease state(s) that are also disease(s) or condition(s) in which Th17 is etiologic for cell, tissue and organ damage can include determining dosage and duration of treatment for specific diseases by determining plasma, tissue, cellular and cerebrospinal fluid levels of Th17 or its cytokine IL-17.

Treatment of a disease with a specific aberrant gut microbiome characteristic of that disease in which Th17 is etiologic in the pathology of the disease state can include measuring the patient's plasma or serum levels and/or cerebrospinal, tissue or cellular levels of Th17, determining whether the patient's Th17 levels are elevated, and administering an oral Type I Interferon in an amount that is effective for alleviating or preventing symptoms of the Th17-led immune attack in the patient.

Determining whether the patient's Th17 levels are elevated can include comparing a baseline Th17 level of the patient before the patient became symptomatic or developed the disease, and comparing that baseline Th17 level to the Th17 level after the patient became symptomatic or developed the disease. Determining whether the patient's Th17 levels are elevated can also include comparing the patient's Th17 levels to a range of Th17 levels found in a healthy population. The range of Th17 levels found in a healthy population is generally thought to be ______ in human plasma. Th17 levels can be measured weekly, every two weeks, every three weeks, every four weeks, every six weeks, every eight weeks, or at longer intervals.

Oral Type I interferons and their analogs can be formulated, administered and/or delivered orally as a suspension, solution, syrup, powder, tablet, capsule, pill, lozenge, or sachet, or other means of oral delivery. Oral formulations can be chosen from those reviewed in the literature (see Adejare A, ed. (2020), Remington: The Science and Practice of Pharmacy, 23rd Ed. (Academic Press, Cambridge, MA) Publisher: Academic Press). The oral formulations can include standard carriers such as pharmaceutical grades of mannitol, lactose, starch, magnesium stearate, sodium saccharine, cellulose, magnesium carbonate, or gelatin as well as excipients such as binders, coatings, diluents, disintegrants, lubricants, and colorants, as described in Remington. The oral formulations can include various liquid solutions or suspensions for oral delivery of the Type I interferons and their analogs.

Amino acid sequences of Type I Interferons are represented by the amino acid sequences of SEQ ID NOS: 5-12.

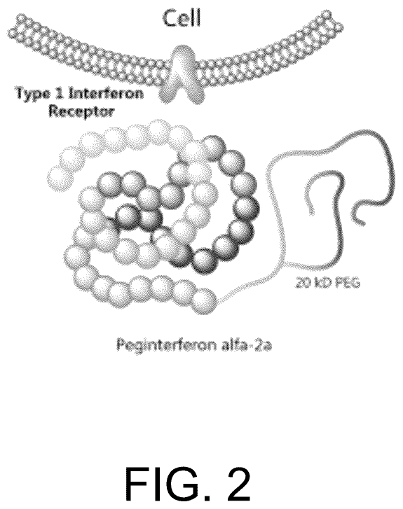

The amino acid sequence of human Interferon Alpha 2a is represented by the amino acid sequence of SEQ ID NO: 5 and SEQ ID NO: 6, where SEQ ID NO: 6 is a pegylated form. An illustration of pegylated Interferon Alpha 2a is shown in FIG. 2.

The amino acid sequence of human Interferon Alpha 2b is represented by the amino acid sequence of SEQ ID NO: 7 and SEQ ID NO: 8, where SEQ ID NO: 8 is a pegylated form.

The amino acid sequence of human Interferon Beta 1a is represented by the amino acid sequence of SEQ ID NO: 9.

The amino acid sequence of human Interferon Beta 1b is represented by the amino acid sequence of SEQ ID NO:10.

The amino acid sequence of human Interferon Gamma is represented by the amino acid sequence of SEQ ID NO:11.

The amino acid sequence of recombinant Interferon Tau is represented by the amino acid sequence of SEQ ID NO: 12.

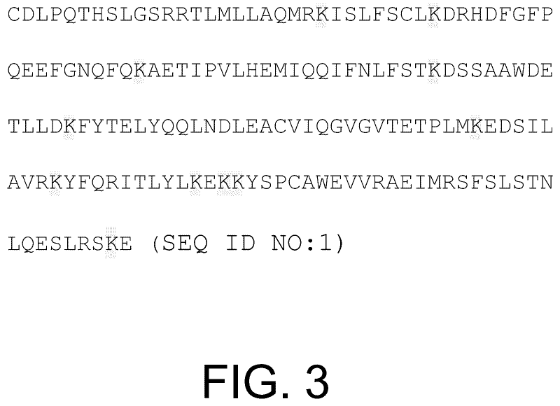

The following modifications may be incorporated to form effective analogs of interferons to improve stability and function. For example, there are 11 positions in Interferon Alpha 2a that can be modified as shown in FIG. 3 by highlighted K, indicating locations of the lysine in Interferon Alpha 2a.

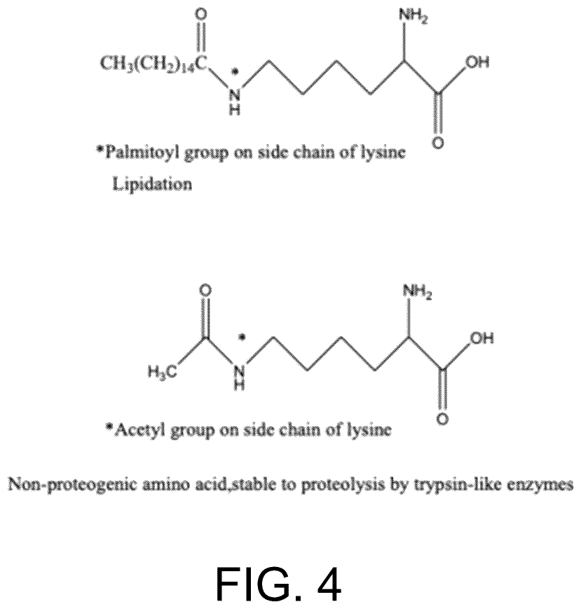

Lysine can be modified in ways including a palmitoyl group or an acetyl group on side chain of lysine to improve stability as shown in FIG. 4 for the interferons represented by SEQ ID NOS: 5-12.

Based on the findings of this invention, usage, and further modifications of Type I interferons for oral use for inhibition of Th17 include novel modifications that maintain biological profile including ability to sustain degradation in pH of 2.0. For example, in evaluating the interferon alpha 2a, opportunities for chemical modification include capping the C and N termini and lipidation of lysine and aspartic/glutamic acid resulting in the introduction of a palmitoyl group such as palmitic acid to lysine or 16-aminopalmitic acid to aspartic or glutamic acid or capping of lysine and aspartic acid with a carboxyl or glutamic acid by the introduction of an amino alkyl group.

According to some embodiments, Type I interferons and their analogs can be given at oral doses of less than 10,000 IU per day, including 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500 IU per day, including doses at any range encompassing these values, such as 100 to 500 IU, 100 to 5000 IU, 500 to 5000 IU, 500 to 2500 IU, 500 to 7500 IU, 1000 to 9000 IU, 500 to 9000 IU, 1000 to 5000 IU, 750 to 2500 IU, and 700 to 7000 IU per day.

According to embodiments, Type I interferons and their analogs can be administered when Th17 levels increase over a baseline measurement in a single patient, such baseline measurement occurring when the patient is asymptomatic, such as 10%, 20%, 25%, 30%, 35%, 40%, 45%, 50%, 55%, 60%, 65%, 70%, 75% or more over baseline, or 1×, 2×, 3×, 4×, 5×, 6×, 7×, 8×, 9×, 10× or more over baseline, or exceed a normal range found in healthy patients, such as exceeding the upper limit of the range, or exceeding one standard deviation of normal, two standard deviation of normal, or three standard deviations of normal.

According to some embodiments, Type I interferons and their analogs can be administered over 1 week, 2 weeks, 3 weeks, 4 weeks, 5 weeks 6 weeks, 7 weeks, 8 weeks, 9 weeks, 10 weeks, three months, four months, five months, six months, seven months, eight months, nine months, ten months, 11 months, 12 months, or longer, with periodic measurement of Th17 levels by ELISA assay or flow cytometry every week, two weeks, four weeks, six weeks, or eight weeks, with dosages of the Type I interferons adjusted according to change in Th17 levels from the previous measurement. Dosage adjustment can be initiated such that the dosage of Type I interferon or analog is reduced when Th17 levels decrease 5%, 10%, 15%, 20%, 25%, 30%, 35%, 40%, 45%, 50%, 55%, 60%, 65%, 70%, 75% or more from the previous measurement, or according to the clinical judgement of the physician administering the Type I interferon or analog.

Conditions in which Th17 involvement is implicated and which may be amenable to the therapeutic and prophylactic methods of the invention include Type I diabetes, autism spectrum disorder, schizophrenia, Parkinson's disease, amyotrophic lateral sclerosis (ALS), Alzheimer's disease, macular degeneration, multiple sclerosis, psoriasis, rheumatoid arthritis, systemic lupus erythematosus, autoimmune hemolytic anemia, autoimmune thrombocytopenia, Guillain-Barre syndrome and inflammatory bowel disease, as well as various cancers, including cholangiocarcinoma, breast and colon cancer. Th17 levels can be measured by ELISA or flow cytometry in conjunction with monitoring of patient symptoms to determine therapeutic efficacy of Type I interferons and doses adjusted according to therapeutic response.

Type I interferons and their analogs can be co-administered with therapeutic agents appropriate for the condition treated at their normal dosages, such as insulin and other diabetes medications, chemotherapeutic agents, anti-inflammatory agents, and neuroleptics.

### EXAMPLES

### Example 1

A patient with a family history of Type 1 Diabetes has two siblings with the disease. The patient has tested positive for IA-2A, GAD and ZnT8 antibodies with family concern about the patient developing acute life-threatening diabetic ketoacidosis, as was the presentation of one of the siblings. Based upon monitoring of hemoglobin A1C, the patient's levels are rising into the prediabetes range, but there is no recommendation for treatment for insulin by the physician since the patient's blood glucose levels remain in the normal range. Recommendations are made to monitor Th17 in the plasma via standard ELISA assay kit every 8 weeks with comparison of the patient's own levels against healthy controls. At the point in time in which the patient's IL-17 level is outside two standard deviations of normal, after weighing the risks and benefits with their being a 100% risk of the patient developing diabetes over the next 5 years, a decision is made to begin 5000 IU of an oral analog of interferon alpha-2a daily at bedtime with measurements of Th17 levels initially weekly for 4 weeks then measurements made every 2 weeks until the time when the patient's Th17 levels reach into the upper limits of normal range among healthy individuals. The dosage of interferon beta-1a, is then reduced to 1000 IU given orally daily and weekly levels of Th17 are measured to ensure that IL-17 levels remain in the normal range for a period of 4 weeks, at which time the dosage is reduced to 500 IU daily, with laboratory studies checked monthly.

### Example 2

A patient presents with symptomatic relapsing-remitting multiple sclerosis after being disease free for six months prior to presentation. Measurement is made of the patient's plasma Th17 levels, which are found to five and fold higher levels, respectively than when the patient was asymptomatic. The patient is given 5000 IU of an analog oral interferon beta-1b in liquid solution that is taken daily at bedtime. Measurements of IL-17 levels are measured initially weekly for 4 weeks then measurements made every 2 weeks until the time when both the patients' symptoms have remitted and the patient's levels of Th17 barely reaching the upper limits of normal range, based upon the patient's previous levels when in remission. The dosage of interferon beta 1a, is then reduced to 1000 IU given orally daily and weekly levels of Th17 are measured to ensure that Th17 levels remain in the normal range without worsening of clinical symptomatology for a period of 4 weeks, at which time the dosage is reduced to 500 IU daily, with laboratory studies check every 2 weeks. Treatment of 500 IU of the analog interferon beta-1b is continued until there is consistent remission for 12 months at which time, the dosage may be reduced by half and given daily at bedtime. The lower dosage should be continued for the duration of the remission. Oral interferon beta-1b in liquid solution may be used in combination or separately with other medications for multiple sclerosis.

### Example 3

A patient who has schizophrenia presents with acute psychosis. The patient's levels of Th17 are noted to be significantly elevated over that during the time the patient was in remission and that of healthy controls, at which time the patient was not treated with an interferon. The levels of Th17 are noted to be five times above the upper limit of normal healthy controls and Th17 is noted to be ten times higher than normal healthy controls. Patients have typically been given a dose regimen of interferon beta-1a given orally at 150 IU orally three times daily, initially, dosing then adjusted based upon Th17 levels into the normal range with maintenance of the lowest possible dosage for absence of symptoms with Th17 levels into the normal range. The titration will be held at the point in time when the Th17 levels are within the normal range with regression of symptoms with measurements of Th17 continued every 2 weeks with maintenance of dosages for 4 weeks at the dosage in which levels are within the normal range without symptomatology.

### Example 4

A patient is diagnosed with Amyotrophic Lateral Sclerosis (ALS) with symptomatology. Th17 is measured and then 1000 IU of oral interferon beta-1a is initiated with the dosage given at bedtime in an oral solution. This dosage is continued and may be used with other treatment modalities. Levels of Th17 are measured monthly and evaluated along with neurological symptomatology and stability vs. progression of disease. Based on the levels of Th17, the dosage may be decreased to 500 IU if the Th17 levels are within the normal range or may be increased 2000 IU of oral interferon beta-1a given at bedtime. Levels of Th17 will be measured at 2-week intervals. With stabilization of symptoms and with levels of Th17 within the normal limits, the lowest dosage will be maintained.

### Example 5

A patient with Type 1 Diabetes who may be newly diagnosed or having existing Type 1 is prescribed a tablet of 5000 IU of interferon beta 1a taken at bedtime, alone or in combination with insulin or an islet neogenesis agent such as Peptide Healing Islets of Langerhans (PHIL). The combination pill may be used alone or in combination with insulin. A combination pill of beta-1 interferon may be combined in one capsule, solution, tablet etc., with an oral form of PHIL or an oral or injectable form of insulin. A combination of oral interferon beta-1a is delivered in an oral capsule that may or may not be combined or taken with an islet neogenesis agent, such as Peptide Healing Islets of Langerhans or taken with subcutaneous insulin, and oral interferon beta-1a is delivered daily with measurements of Th17 every two weeks with the goal of tracking Th17 levels into the normal range at which time the dosage will be decreased to 1000 IU daily with checking of Th17 on a monthly basis.

### Example 6

Oral Interferons may be used separate or in combinations to treat all mammals with medical conditions, including infections of all types including but not limited to viral, bacterial, fungal infections, or disease states and cancers for which there is not optimal treatment. Mammals may include, but are not limited to humans, dogs, cats, horses, pigs, and cattle. Oral interferons dosage will be based on the initial Th17 levels and the underlying symptomatology and disease state with doses ranging from 500-5000 IU of interferon at nighttime and used with other medical therapies and treatments for the duration of symptomatology. Based on the severity of the medical condition in the mammal, measurements of Th17 may be checked every two weeks to determine the trend towards that of a healthy mammal and the dosage adjusted accordingly.

1. A method comprising:
administering a Type I interferon or Type I interferon analog orally at a dosage of less than 10,000 IU per day to a human or other mammal suffering from a disease or condition associated with aberrant gut microbiomes and in which Th17 is etiologic in cell, tissue, and organ damage.

2. The method of claim 1, wherein the Type I interferon or Type I interferon analog is administered orally as a suspension, solution, syrup, powder, tablet, capsule, pill, lozenge, or sachet.

3. The method of claim 1, wherein the Type I interferon or Type I interferon analog is administered orally at a dosage in the range of 100 to 5000 IU per day to the human or other mammal.

4. The method of claim 1, wherein the Type I interferon or Type I interferon analog is chosen from alpha, beta, gamma and tau interferons or interferon analogs.

5. The method of claim 1, wherein the disease or condition is chosen from Type I diabetes, autism spectrum disorder, schizophrenia, Parkinson's disease, amyotrophic lateral sclerosis (ALS), Alzheimer's disease, macular degeneration, multiple sclerosis, psoriasis, rheumatoid arthritis, systemic lupus erythematosus, autoimmune hemolytic anemia, autoimmune thrombocytopenia, Guillain-Barré syndrome, inflammatory bowel disease, cholangiocarcinoma, breast cancer and colon cancer.

6. A method comprising:
administering a Type I interferon or Type I interferon analog orally at a dosage of less than 10,000 IU per day to a human or other mammal thereby preventing a disease or condition associated with aberrant gut microbiomes and in which Th17 is etiologic in cell, tissue, and organ damage.

7. The method of claim 6, wherein the Type I interferon or Type I interferon analog is administered orally as a suspension, solution, syrup, powder, tablet, capsule, pill, lozenge, or sachet.

8. The method of claim 6, wherein the Type I interferon or Type I interferon analog is administered orally at a dosage in the range of 100 to 5000 IU per day to the human or other mammal.

9. The method of claim 6, wherein the disease or condition is chosen from Type I diabetes, autism spectrum disorder, schizophrenia, Parkinson's disease, amyotrophic lateral sclerosis (ALS), Alzheimer's disease, macular degeneration, multiple sclerosis, psoriasis, rheumatoid arthritis, systemic lupus erythematosus, autoimmune hemolytic anemia, autoimmune thrombocytopenia, Guillain-Barré syndrome, inflammatory bowel disease, cholangiocarcinoma, breast cancer and colon cancer.

10. A Type I interferon analog designed to resist degradation in the gastrointestinal tract and capable of inhibiting Th17 in the gastrointestinal tract when administered orally, the Type I interferon analog modified by one or more of:
lipidation of one or more amino acids with introduction of a palmitoyl group, such as lipidation of one or more lysine residues by palmitic acid or lipidation of one or more aspartic acid or glutamic acid residues by 16-aminopalmitic acid;
acetylation of one or more lysine residues;
capping of one or more lysine or aspartic acid residues with a carboxyl group;
capping of one or more glutamic acid residues by the introduction of an amino alkyl group; and
capping the C and N termini; and
pegylation.

11. The Type I interferon analog of claim 10, wherein the Type I interferon analog is formulated as a suspension, solution, syrup, powder, tablet, capsule, pill, lozenge, or sachet.

12. The Type I interferon analog of claim 10, wherein the Type I interferon analog is formulated at a dosage of less than 10,000 IU.

13. The Type I interferon analog of claim 10, wherein the Type I interferon analog is formulated at a dosage in the range of 100 to 5000 IU.

14. The Type I interferon analog of claim 10, wherein the Type I interferon analog is chosen from alpha, beta, gamma and tau interferon analogs.

15-25. (canceled)
